(19) United States
(12) Patent Application Publication
(10) Pub. No.: US 2007/0156096 A1
(43) Pub. Date: Jul. 5, 2007

(54) Puncture device

(75) Inventors: Yutaro Sonoda, Ashigarakami-gun (JP); Tetsuro Kawanishi, Ashigarakami-gun (JP)

Correspondence Address: BUCHANAN, INGERSOLL & ROONEY PC, POST OFFICE BOX 1404, ALEXANDRIA, VA, 22313-1404, US

(73) Assignee: TERUMO KABUSHIKI KAISHA, Tokyo (JP)

(21) Appl. No.: 11/594,920

(22) Filed: Nov. 9, 2006

Related U.S. Application Data
(60) Provisional application No. 60/735,176, filed on Nov. 10, 2005.

(51) Int. Cl. A61M 5/32

(52) U.S. Cl. 604/174

(57) ABSTRACT

To provide a puncture device in which a puncture needle is stuck into a predetermined region certainly and an indwelling device can be indwelled, the puncture device is provided for puncturing skin (dermis) by a puncture needle. This puncture device is provided with a puncture needle, an indwelling device indwelled in the skin by the puncture needle, a skin deforming mechanism for deforming the skin which is provided with a fixing portion having a space in an inside constituted by a bottom face portion and a peripheral wall portion 5b. In addition, an insertion portion for inserting the puncture needle is formed at the peripheral wall portion, the puncture needle is inserted from the insertion portion, and the skin deformed into the space by the skin deforming mechanism is adapted to be punctured.

### TECHNICAL FIELD

The present invention relates to a puncture device and relates to a puncture device which is used to puncture a predetermined region, for example, a dermis by puncturing from a skin surface.

### BACKGROUND ART

It is known that the dermis has a high density of capillary blood vessel when compared with an epidermis or a subcutis and also a lymph vessel end exists therein, so that in particular, a medical agent injected thereto directly is shifted to a blood vessel or a lymph vessel and an absorption speed thereof being absorbed in the body fluid is speedy. In particular, it is possible in the dermis to make a medical agent using a macromolecular substance such as hormone, antibody drug, cytokine and the like to be absorbed into the blood efficiently. Also, it is known that the dermis is a place of efficient immunity and it is possible to make a saving of applied dose of vaccine or to strengthen sensitization of a weak vaccine.

Also, it is known for adult human beings that the dermis exists approximately in a certain amount of depth from the body surface (surface of stratum corneum). In other words, this fact means in case of injecting a medical agent into the dermis that it is possible to use a puncture needle having the same length (depth) with respect to these human beings.

Generally, the width of the dermis is around 1 mm to 4 mm (average value is 1 mm to 2 mm) if the vertical direction with respect to the body surface is made to be a reference and also, as shown in FIG. 12, the dermis exists in the skin so as to be sandwiched between an epidermis E which includes a stratum corneum and has width of around 0.06 mm to 0.1 mm and a subcutis S.

Accordingly, it is difficult to insert a tip portion of the puncture device, for example, a needlepoint of the puncture needle accurately to the dermis which exists between the epidermis and the subcutis and if the needlepoint is inserted erroneously to the subcutis or the like, there occurs a problem such that a medical agent cannot be absorbed efficiently.

In recent years, for example, it is attempted that the macromolecular medicine mentioned above is administered continually or by one-shot into the dermis as a target and in such a case, in particular, the above-mentioned problem becomes conspicuous.

Here, a hypodermic injection device is known in which the length of the puncture needle to be inserted into the body is defined in order to inject a medical agent to the dermis in the body (Patent Document 1). In addition, a medicinal solution injection device is also known in which the depth (insertion depth) of the puncture needle to be inserted into the skin is defined to be a predetermined length in order to inject a medical agent into a specific layer which exists in the skin and the puncture needle is inserted into the skin from the vertical direction with respect to the body surface (Patent Document 2).
[Patent Document 1] Japanese Patent Laid-open No. 2001-137343
[Patent Document 2] Japanese Patent Laid-open No. 2005-87519

### DISCLOSURE OF THE INVENTION

### Problem to be Solved by the Invention

However, there is employed in these devices a constitution in which the puncture needle is inserted into the skin from the vertical direction with respect to the body surface. In this case, if it is attempted to puncture with the puncture needle, the whole skin is sunk in elastically so as not to be punctured and also, even if it is punctured, it sometimes happens that the needlepoint cannot reach the dermis.

Also, when the puncture needle is inserted perpendicularly with respect to the dermis, the depth (insertion depth) of the puncture needle in the dermis becomes short and, for example, in such a case where some kind of impact or the like is added from the outside, there occurs a problem in which the puncture needle during injecting a medical agent drops out from the dermis.

Further, in case of using these devices, the distance from an insertion aperture of the puncture needle which is formed on the surface of the dermis (boundary portion of epidermis and dermis) until a medical agent releasing opening which exists at the tip of the puncture needle becomes short in the dermis in which there happens a problem that the medical agent injected to the dermis from the medical agent releasing opening is to leak from the insertion aperture to the outside of the dermis (epidermis).

In consideration of the above-mentioned problems, an object of the present invention lies in providing a puncture device in which a puncture needle can be stuck certainly to a predetermined region of a skin and an indwelling device can be indwelled.

### Means for Solving the Problem

Such an object is accomplished according to the present invention of the following (1) to (9).

(1) In a puncture device puncturing a skin by a puncture needle, a puncture device comprising:

a puncture needle;

an indwelling device indwelled in the skin by said puncture needle;

a skin deforming means for deforming the skin which is provided with a fixing portion having a space in an inside constituted by a bottom face portion and a peripheral wall portion, characterized by being constituted such that

an insertion portion for inserting the puncture needle is formed at said peripheral wall portion,

said puncture needle is inserted from said insertion portion, and

the skin deformed into said space by said skin deforming means is to be punctured.

(2) The puncture device according to said (1), characterized in that said skin deforming means makes deformation so as to uplift said skin.

(3) The puncture device according to said (1), characterized in that said skin deforming means makes deformation so as to push down said skin.

(4) The puncture device according to said (1), characterized in that a retaining portion for retaining and fixing the indwelling device is formed at said peripheral wall portion.

The puncture device according to said (4), characterized in that said retaining portion is formed at a position which becomes symmetrical to said insertion portion.

(6) The puncture device according to said (1), characterized in that said indwelling device is a catheter.

The puncture device according to said (6), characterized in that said catheter has at least more than one medical agent supplying opening.

(8) The puncture device according to said (1), characterized in that said indwelling device is a catheter type detection sensor.

(9) In a puncture device puncturing a skin by a puncture needle, a puncture device comprising:

a puncture needle;

an indwelling device indwelled in the skin by said puncture needle;

a fixing portion having a space inside and being constituted by a bottom face portion and a peripheral wall portion; and

a suction means for sucking air of said space, characterized by being constituted such that

an insertion opening for inserting the puncture needle is formed at said peripheral wall portion,

said skin is uplifted into said space using said suction means by sucking the air of said space and by depressurizing said space,

said puncture needle is inserted from said insertion opening, and

said uplifted skin is to be punctured.

The above-described object is accomplished also by the invention of the following (10).

(10) In a puncturing method puncturing a skin by a puncture needle, a puncturing method using:

a puncture needle;

an indwelling device indwelled in the skin by said puncture needle;

a fixing portion having a space in the inside and constituted by a bottom face portion and a peripheral wall portion; and

a skin deforming means for deforming the skin, characterized by being constituted such that

said puncture needle is inserted into an insertion portion formed at said peripheral wall portion for inserting a puncture needle, and the skin deformed in said space by said skin deforming means is to be punctured.

### Effect of the Invention

According to the present invention, it is possible for a puncture needle to reach a predetermined region certainly after being stuck from a skin. Also, it is possible for an indwelling device to be indwelled in a predetermined region certainly.

### BRIEF DESCRIPTION OF DRAWINGS

FIG. 1 is a perspective view of a puncture device of a first exemplified embodiment of the present invention.

FIG. 2 is a cross-section view of the puncture device of the first exemplified embodiment of the present invention.

FIG. 3 is a diagram (cross-section view) for explaining the usage of the puncture device shown in FIG. 2.

FIG. 4 is a diagram (cross-section view) for explaining the usage of the puncture device shown in FIG. 2.

FIG. 5 is a cross-section view of a puncture device of a second exemplified embodiment of the present invention.

FIG. 6 is a diagram (cross-section view) for explaining the usage of the puncture device shown in FIG. 5.

FIG. 7 is a diagram (cross-section view) for explaining the usage of the puncture device shown in FIG. 5.

FIG. 8 is a modified example of a puncture device of the present invention.

FIG. 9 is a cross-section view of a puncture device of a third exemplified embodiment of the present invention.

FIG. 10 is a diagram (cross-section view) for explaining the usage of the puncture device shown in FIG. 9.

FIG. 11 is a diagram (cross-section view) for explaining the usage of the puncture device shown in FIG. 9.

FIG. 12 is a cross-section view showing a general skin structure.

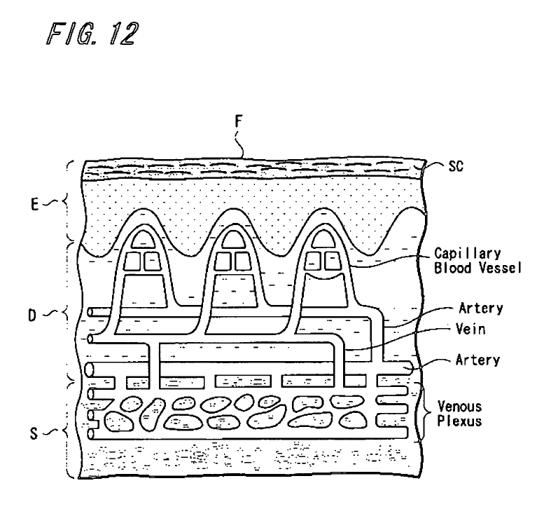

### BEST MODE FOR CARRYING OUT THE INVENTION

Hereinafter, it will be explained with respect to best modes for practicing the puncture device of the present invention with reference to the drawings, wherein it should be noted that the present invention is not limited by the following modes.

### FIRST EXEMPLIFIED EMBODIMENT

First, it will be explained with respect to a first exemplified embodiment of a puncture device 1 of the present invention.

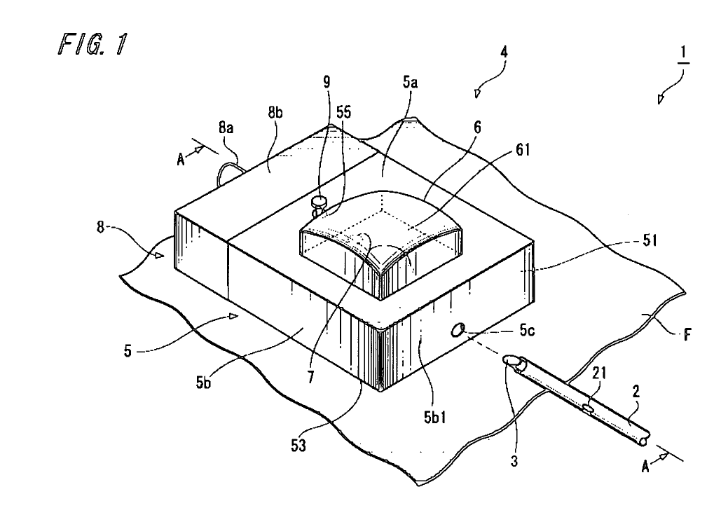

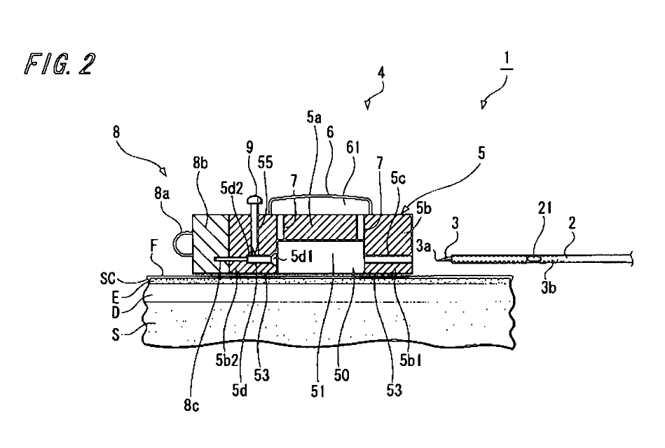

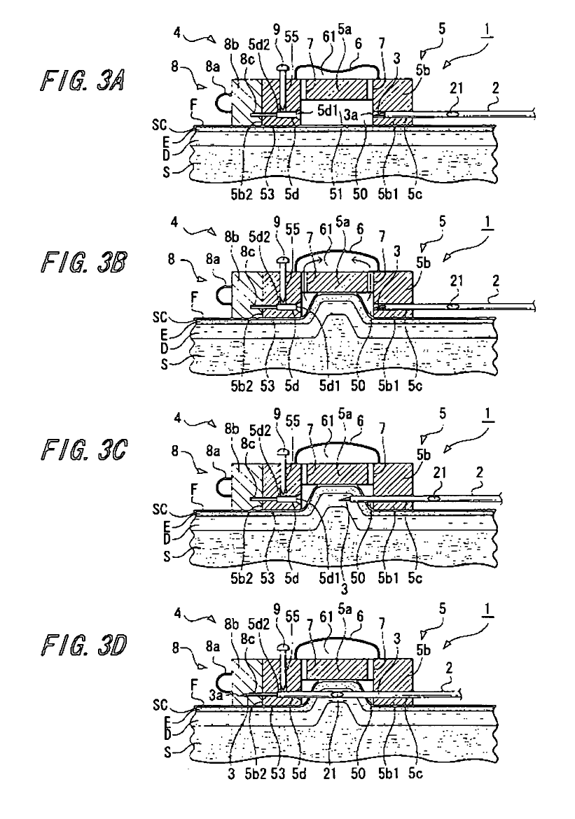

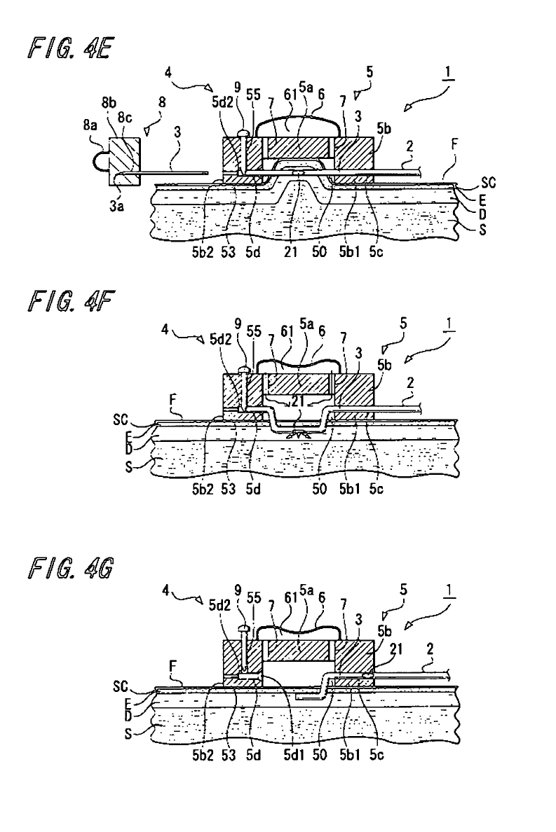

FIG. 1 is a perspective view showing a first exemplified embodiment of a puncture device 1 of the present invention. Also, FIG. 2 is a cross-section view showing a first exemplified embodiment of the puncture device 1 of the present invention. Also, FIG. 3 and FIG. 4 are diagrams (cross-section views) for explaining usage of the puncture device shown in FIG. 2. It should be noted that it will be explained hereinafter on an assumption that the upside in FIG. 1 to FIG. 4 is made to be “rear end” and the downside thereof is made to be “tip”.

The puncture device shown in FIG. 1 is constituted by a catheter 2, a puncture needle 3 and skin deforming means 4.

The skin deforming means 4 is constituted by a fixing portion 5 and a suction means 6.

The fixing portion 5 is provided with a tabular bottom face portion 5a and a peripheral wall portion 5b having four side surface plates positioned adjacent to this bottom face portion 5a.

Also, as shown in FIG. 2, there is formed on the tip side of the fixing portion 5 with an opening portion 50 and this opening portion 50 is constituted so as to be opened up toward the body surface F.

This fixing portion 5 has a constitution in which a space 51 formed in the inside thereof is to be hermetically-closed (sealed to be airtight) by pressing the opening portion 50 to the skin (body surface F). It should be noted that there are arranged, on the lower side of the body surface F, a stratum corneum SC, an epidermis E including the stratum corneum SC, a dermis D and a subcutis S in the form of laminae.

There can be cited for the constituent material of the fixing portion 5 various kinds of resins such as polyethylene, polypropylene or the like and the fixing portion 5 is manufactured by a forming process inpouring these resins into a die which has a predetermined shape or the like.

A fixing surface 53 composed of a plane having a predetermined area is formed on the tip side of the peripheral wall portion 5b. It is possible owing to the fixing surface 53 to improve airtightness of the space 51 when pressing the opening portion 50 to the body surface F. Also, by making the area of the fixing surface 53 to be large, it is possible to improve stability of the fixing portion 5 with respect to the skin.

On one side surface plate 5b1 of the peripheral wall portion 5b, an insertion opening 5c as an insertion portion for inserting the catheter 2 to be described hereinafter is formed so as to become in parallel with the fixing surface 53. The insertion opening 5c is formed so as to pass through the side surface plate 5b1 from the outside the fixing portion 5 toward the space 51.

Also, on the side surface plate 5b2 facing to the side surface plate 5b1 and at a position symmetrical to the insertion opening 5c, a fixing opening 5d as a retaining portion for fixedly retaining the catheter 2 is formed so as to become in parallel with the fixing surface 53. Also, the fixing opening 5d is formed so as to pass through the side surface plate 5b2 from the space 51 toward the outside of the fixing portion 5.

There is formed on the space 51 side of the fixing opening 5d with a taper portion 5d1 so as to be directed to the space 51. Thereby, it is possible to make it easy for a needlepoint 3a of the puncture needle 3 to be inserted into the fixing opening 5d.

Also, there is formed on the opposite side of the fixing opening 5d to the space 51 with a step portion 5d2 whose opening diameter is different. This step portion 5d2 is formed such that the diameter on the space 51 side becomes small. It is possible by such a step portion 5d2 to make the inserted puncture needle 3 to be separated more easily from the catheter 2.

Also, there is formed on the outer surface (upper surface) of the side surface plate 5b2 with a pin hole 55 which is in communication with the fixing opening 5d. A fixing pin 9 is engaged with the pin hole 55 so as to be movable in the upper and lower direction.

There is provided on the outer surface of the side surface plate 5b2 with a mounting/dismounting member 8 detachably with respect to the side surface plate 5b2. The mounting/dismounting member 8 is constituted by a handle 8a and a main body 8b. The main body 8b is mounted on the outer surface of the side surface plate 5b2 and there is formed on the mounting surface of the main body 8b with an insertion opening 8c which coincides with the fixing opening 5d.

There is formed on the bottom face portion 5a with two through-holes 7 as flow channels of air. The through-holes 7 are positioned at the upper portion of the space 51 and are formed so as to pass through the bottom face portion 5a from the space 51 toward the rear end side.

An elastic body 6 having a hollow portion 61 as a suction means is firmly-fixed (fixed) on the outer surface (upper surface) of the bottom face portion 5a.

The elastic body 6 has a cube shape and is constituted by an elastically deformable member. Also, the hollow portion 61 is formed inside the elastic body 6. It should be noted that a cube shaped body is used as an elastic body in this exemplified embodiment, but the shape of the elastic body is not limited by this and, for example, a dome shape may be used.

Also, the hollow portion 61 of the elastic body 6 is constituted so as to be in communication with the space 51 of the fixing portion 5 through the through-holes 7.

By pressing the opening portion 50 of the fixing portion 5 to the body surface F in a state in which the elastic body 6 is compressed and by releasing the compressed state of the elastic body 6, air in the space 51 is sucked into the hollow portion 61 of the elastic body 6, the space 51 is depressurized and it is possible to deformed the skin so as to be uplifted.

Also, by making the elastic body 6 to be in a compressed state again and by sending the air in the hollow portion 61 to the space 51 of the fixing portion 5, it is possible to pressurize the space 51 and to deform the skin so as to be pushed down.

For the constituent material of the elastic body 6, there can be cited various kinds of rubber materials such as natural rubber, isoprene rubber, butadiene rubber, styrene-butadiene rubber, nitrile rubber, chloroprene rubber, butyl rubber, acrylic rubber, ethylene-propylene rubber, hydrin rubber, urethane rubber, silicone rubber, fluoro-rubber and the like or various kinds of thermoplastic elastomers such as styrene series, polyolefin series, polyvinylchloride series, polyurethane series, polyester series, polyamide series, polybutadiene series, fluoro-rubber series and the like.

It should be noted for the suction means that it is allowed to employ such a constitution wherein, for example, a syringe is fixed at an edge portion of a tube which is made to be in communication with the space 51 and the air in the space 51 is to be sucked by the operation of the syringe.

The puncture needle 3 is constituted by a linear body portion 3b and a tapered needlepoint portion 3a.

The body portion 3b of the puncture needle 3 is inserted into a hollow portion of the catheter 2 to be described hereinafter and is constituted such that the needlepoint portion 3a is to be projected from the catheter 2.

The length of the body portion 3b is around 5.0 mm to 50.0 mm, and while the outer diameter of the puncture needle 3 becomes a little bit different depending on the use application of the puncture device 1 or the like, it is preferable to be around 0.1 mm to 4.0 mm and in particular, it is preferable to be around 0.5 mm to 2.0 mm.

For the constituent material of the puncture needle 3, there can be cited, for example, a metal material such as stainless steel, aluminum or aluminum alloy, titanium or titanium alloy or the like. Also, the puncture member 3 is manufactured, for example, by plastic working.

The catheter 2 as an indwelling device is constituted by an elongated member having flexibility. Also, there is formed in the inside thereof with a hollow portion (flow channel) along the longitudinal direction.

It is preferable for the length of the catheter 2, although it is not limited in particular, to be selected usually as 50 mm to 1000 mm and more preferably to be selected as 80 mm to 700 mm. Also, it is preferable for the outer diameter of the catheter 2, although it is not limited in particular, to be selected usually as 0.1 mm to 4.0 mm and more preferably to be selected as 0.5 mm to 2.0 mm.

Also, there is formed at a position apart from the tip portion of the catheter 2 by a predetermined distance with a medical agent supplying opening 21. It is allowed to make the medical agent supplying opening 21 to be positioned at a tip portion or an intermediate portion of the catheter 2 and it is allowed to form a plurality of medical agent supplying openings.

A liquid transmission pump (not shown) is connected to one edge portion of the catheter 2 and a medical agent is supplied to the medical agent supplying opening 21 by way of the hollow portion.

There can be cited for the medical agent, for example, remedy for injection or medicinal solution using a macromolecular substance such as hormone, antibody drug, cytokine, vaccine or the like.

It should be noted for the indwelling device that it is not limited by the catheter supplying the medical agent, but it may be also allowed to employ a fiber cable catheter with a sensor having sensor function, for example, a sensor for saccharide measurement provided with an optical fiber disclosed in Japanese Patent Laid-open No. H08-107890. Also, a plurality of sensors may be provided for one catheter.

Also, in case of the sensor for the saccharide measurement, it is allowed to use a fluorescent sensor including a fluorescent dye composed of a phenyl boron derivative.

Also, in case of using amicrodialysis sensor for the fiber cable catheter with a sensor, a portion of the catheter is formed by a dialysis membrane.

In case of the catheter having sensor function, a measuring apparatus is connected to the edge portion of the catheter and a state in the skin will be measured by the sensor.

In case of using the fluorescent sensor, a fluorescent measuring device is used for the measuring apparatus.

In case of using the microdialysis sensor, a substance to be measured in dialysate solution of the catheter will be measured by the sensor.

It should be noted that it is allowed for one catheter to be provided with both functions of the medical agent supply function and the sensor function.

Next, it will be explained by using FIG. 3 and FIG. 4 with respect to usage (operation) of the puncture device 1 of this exemplified embodiment.

First, the fixing portion 5 is mounted on a predetermined position of the body surface F (skin). At that time, it is allowed to provide an adhesive film or the like on the fixing surface 53 in order to improve stability of the fixing portion 5 with respect to the body surface F.

In this case, as shown in FIG. 3(a), the fixing portion 5 is mounted on the body surface F in a state in which the elastic body 6 is deformed by being grasped with fingers or the like and the air in the hollow portion 61 of the elastic body 6 is exhausted.

Concurrently with this, the catheter 2 inserted and attached with the puncture needle 3 is inserted into the insertion opening 5c formed on the peripheral wall portion 5b of the fixing portion 5 and is retained in the inside of the peripheral wall portion 5b. Alternatively, a constitution may be employed in which the catheter 2 mounted with the puncture needle 3 is retained beforehand in the inside of the peripheral wall portion 5b.

Next, as shown in FIG. 3(b), the fingers is released from the elastic body 6 and the compressed state of the elastic body 6 is released in which the air in the space 51 of the fixing portion 5 is sucked into the hollow portion 61 through the through-hole 7 as shown by arrows. In this exemplified embodiment, a plurality of through-holes 7 are provided, so that it is possible to suck the air in the space 51 evenly toward the hollow portion 61.

Thereby, the space 51 is depressurized and the skin (body surface F) on the inner side of the opening portion 50 is uplifted into the space 51. More specifically, the epidermis E including the stratum corneum SC, the dermis D and the subcutis S are uplifted to the vertical direction with respect to the body surface F. The height of the space 51 is set such that the position of the uplifted dermis D becomes the same height as the position of the insertion opening 5c provided on the peripheral wall portion 5b.

It should be noted that it may be constituted such that an adhesive film or the like is provided on the body surface F side of the bottom face portion 5a and the uplifted body surface F is to be adhesively fixed.

At that time, the body surface F is uplifted in a state in parallel with the bottom face portion 5a, so that the dermis D is also uplifted into the space 51 of the fixing portion 5 in a state in parallel with the bottom face portion 5a.

Next, the puncture needle 3 mounted onto the catheter 2 is inserted into the space 51 and punctured to the uplifted skin.

At that time, the puncture needle 3 is stuck to the skin in a state in parallel with the bottom face portion 5a, so that, as shown in FIG. 3(c), the puncture needle 3 is stuck in the skin in a state in parallel with the dermis D.

From this state, the catheter 2 is further inserted and, as shown in FIG. 3(d), the puncture needle 3 is inserted into the insertion hole 8c of the mounting/dismounting member 8 mounted on the side surface plate 5b2 of the peripheral wall portion 5b and the catheter 2 is inserted into the fixing opening 5d provided on the side surface plate 5b2 of the peripheral wall portion 5b. At that time, the medical agent supplying opening 21 provided in the catheter 2 is positioned in the vicinity of the central portion of the dermis D uplifted into the space 51.

Also, the tip of the catheter 2 is latched in the fixing hole 5d by means of the step portion 5d2 formed in the fixing hole 5d.

Next, as shown in FIG. 4(e), the handle 8a of the mounting/dismounting member 8 is grasped by fingers or the like and the main body 8b is detached from the side surface plate 5b2. Thereby, the puncture needle 3 is separated from the catheter 2 in a state of being clipped by the insertion hole 8c and is pulled out from the fixing portion 5.

Next, the fixing pin 9 is pushed down to the tip direction, the tip portion of the fixing pin 9 is engaged with the catheter 2 inserted in the fixing opening 5d and the tip portion of the catheter 2 is fixed in the fixing opening 5d.

Next, as shown in FIG. 4(f), the elastic body 6 is depressed and deformed by a finger or the like concurrently. Thereby, the air in the hollow portion 61 of the elastic body 6 is sent to the space 51 of the fixing portion 5 through the through-holes 7. Thereby, the space 51 in the fixing portion 5 is pressurized, so that owing to this force, the skin uplifted in the space 51 is deformed so as to be pushed down.

In this state, the medical agent is sent to the medical agent supplying opening 21 from the liquid transmission pump which is connected to the edge portion of the catheter 2 through the hollow portion of the catheter 2. Then, the medical agent is injected into the dermis D from the medical agent supplying opening 21.

Here, it is constituted in case of using the catheter type sensor having sensor function such that the sensor is to be positioned at the position of the medical agent supplying opening. In this case, a state in the skin is measured by the measuring apparatus which is connected to the edge portion of the catheter through the sensor.

After the medical agent is injected into the dermis D, as shown in FIG. 4(g), the fixing pin 9 is pulled out to the rear end direction and the engagement of the fixing pin 9 and the catheter 2 is released. Then, the catheter 2 is pulled out from the insertion opening 5c and thereafter, the fixing portion 5 is detached from the body surface F and the puncture operation is completed.

In this manner, according to the puncture device 1 of this exemplified embodiment, in a state in which the opening portion 50 of the fixing portion 5 is pressed to the body surface F the air in the space 51 is sucked by the elastic body 6 so as to depressurize the space 51. Thereby, a puncture operation is carried out in a state in which the skin including the dermis D is uplifted into the space 51, so that it is possible to stick the puncture needle 3 certainly to the skin (dermis D) and it is possible to indwell the catheter 2 certainly in the dermis D.

Also, it is possible to inject a medical agent to the dermis D certainly by means of the catheter indwelled in the skin. Also, it is possible to measure the state of the dermis D certainly by means of the indwelled catheter type sensor.

More specifically, according to the puncture device 1 of the present invention, the catheter is retained by the puncture needle and after the puncture, by taking out only the puncture needle in which the catheter is left, the catheter is to be implanted in the inside of the skin. By implanting a hollow catheter which has an opening at the implanted portion, it is possible to use it for continuous administration of a medical agent or for a purpose of sampling a body fluid component. Also, by implanting a catheter which has a sensor at the implanted portion, it is also possible to carry out measurement of a component in the body.

Further, it is possible to indwell the catheter 2 approximately in a parallel state with the dermis D, so that the insertion depth of the catheter in the inside of the dermis D becomes long and even in a case in which an impact or the like is added from the outside, the catheter 2 during injecting the medical agent can be prevented from dropping out from the dermis D.

Also, the distance from the insertion aperture of the catheter 2, which is formed at a boundary portion between the epidermis E and the dermis D until the medical agent supplying opening 21 becomes long, so that the medical agent once injected into the dermis D from the medical agent releasing opening can be prevented from leaking from the insertion aperture to the epidermis E by being flown back.

### SECOND EXEMPLIFIED EMBODIMENT

Next, it will be explained with respect to a second exemplified embodiment of the puncture device 1 of the present invention.

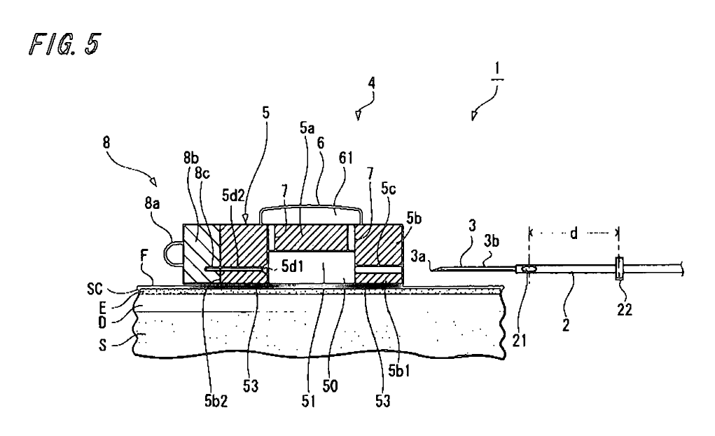

FIG. 5 is a cross-section view showing the second exemplified embodiment of the puncture device 1 of the present invention.

Hereinafter, it will be explained with respect to the puncture device 1 of the second exemplified embodiment mainly about differences from the puncture device of aforesaid first exemplified embodiment and with respect to similar matters, the explanations thereof will be omitted.

In this exemplified embodiment, there is formed at a tip portion of the catheter 2 with the medical agent supplying opening 21. Also, there is mounted at a position apart from the tip portion of the catheter 2 by a predetermined distance d with a ring-shaped positioning member along the circumference of the catheter 2.

Also, the edge portion of the body portion 3b of the puncture needle 3 is inserted and attached in the vicinity of the tip of the catheter 2 and it is in a state in which most portions of the body portion 3b are exposed toward the outside of the catheter 2.

It should be noted that being different from the first exemplified embodiment, the fixing portion 5 is not provided with a member such as the fixing pin 9 or the like which is engaged with the catheter 2. Also, there is not provided at the fixing opening 5d provided on the peripheral wall portion 5b with any step portion or the like.

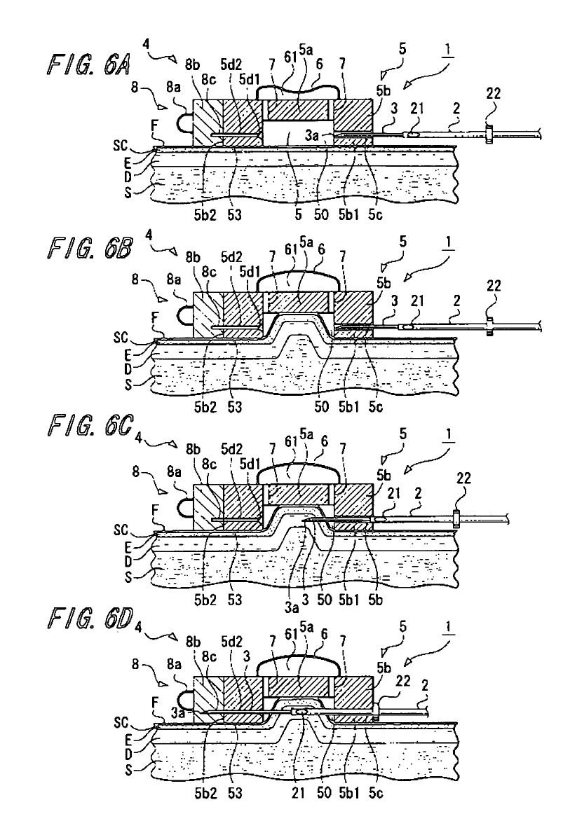

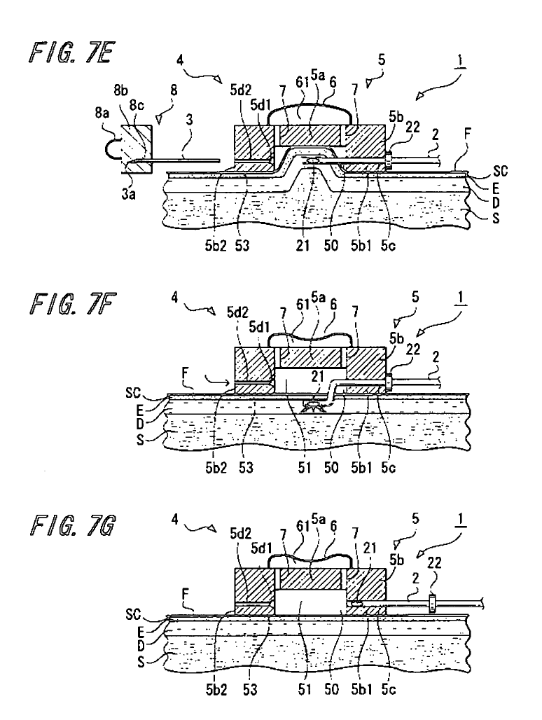

Next, it will be explained by using FIG. 6 and FIG. 7 with respect to usage (operation) of the puncture device 1 of this exemplified embodiment.

First, as shown in FIG. 6(a), the elastic body 6 is deformed by being grasped with fingers or the like and the fixing portion 5 is mounted on the body surface F in a state in which the air in the hollow portion 61 of the elastic body 6 is exhausted.

Concurrently with this, the catheter 2 inserted and attached with the puncture needle 3 is inserted into the insertion opening 5c formed on the peripheral wall portion 5b of the fixing portion 5. In this case, being different from the first exemplified embodiment, only the needlepoint 3a of the puncture needle 3 and a portion of the body portion 3b are retained in the inside of the peripheral wall portion 5b.

Next, as shown in FIG. 6(b), the finger is released from the elastic body 6 and the compressed state of the elastic body 6 is released in which the air in the space 51 of the fixing portion 5 is sucked into the hollow portion 61 through the through-hole 7 as shown by arrows.

Thereby, the space 51 is depressurized and the skin (body surface F) on the inner side of the opening portion 50 is uplifted into the space 51. More specifically, the epidermis E including the stratum cormeum SC, the dermis D and the subcutis S are uplifted to the vertical direction with respect to the body surface F. The height of the space 51 is set such that the position of the uplifted dermis D becomes the same height as the position of the insertion opening 5c provided on the peripheral wall portion 5b.

Next, the puncture needle 3 mounted onto the catheter 2 is inserted into the space 51 and stuck to the uplifted skin.

At that time, the puncture needle 3 is stuck to the skin in a state in parallel with the bottom face portion 5a, so that, as shown in FIG. 6(c), the puncture needle 3 is stuck in the skin in a state in parallel with the dermis D.

From this state, as shown in FIG. 6(d), the catheter 2 is inserted into the fixing portion 5 until the positioning ring 22 provided on the catheter 2 is engaged with the outside wall of the peripheral wall portion 5b.

At that time, the puncture needle 3 is inserted into the fixing opening 5d provided on the side surface plate 5b2 of the peripheral wall portion 5b and into the insertion hole 8c provided on the mounting/dismounting member 8. In this case, being different from the first exemplified embodiment, it never happens that the catheter 2 is to be inserted into the fixing opening 5d.

At that time, the medical agent supplying opening 21 provided at the tip of the catheter 2 is positioned in the vicinity of the central portion of the dermis D uplifted into the space 51.

Next, as shown in FIG. 7(e), the handle 8a of the mounting/dismounting member 8 is grasped by fingers or the like and the main body 8b is detached from the side surface plate 5b2. Thereby, the puncture needle 3 is separated from the catheter 2 in a state of being clipped by the insertion hole 8c and is pulled out from the fixing portion 5.

Next, as shown in FIG. 7(f), the elastic body 6 is depressed and deformed by a finger or the like concurrently and the air in the hollow portion 61 is sent to the space 51 of the fixing portion 5 through the through-holes 7. Thereby, the space 51 is pressurized and the skin uplifted in the space 51 is deformed so as to be pushed down and the skin is made to return to the original state.

In this state, the medical agent is sent to the medical agent supplying opening 21 from the liquid transmission pump which is connected to the edge portion of the catheter 2 through the hollow portion of the catheter 2. Then, the medical agent is injected into the dermis D from the medical agent supplying opening 21.

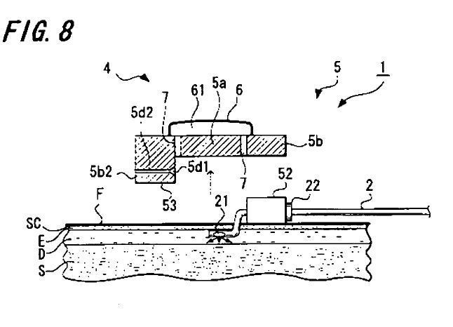

Also, it may be constituted as a modified example of this exemplified embodiment in case of supplying a medical agent to the dermis D such that the fixing portion 5 is to be separable. Specifically, as shown in FIG. 8, the side surface 5b1 of the peripheral wall portion 5b on which the insertion opening 5c is formed is constituted to be separable and it is made to be a state in which this separation portion 52 is to be maintained on the body surface F as being mounted thereon.

In this manner, the separation portion 52 remained on the body surface F fixedly retains the catheter 2 which is on the way of supplying the medical agent, so that it is possible to supply the medical agent to the dermis D stably.

Also, it is possible for a user of the puncture device 1 to directly confirm the state of the catheter 2 placed on the skin, so that it is possible to present a feeling of security to the user.

After the medical agent is injected into the dermis D, as shown in FIG. 7(g), the catheter 2 is pulled out from the insertion opening 5c and the fixing portion 5 is detached from the body surface F and the operation is completed.

According to the puncture device 1 of this exemplified embodiment, even in case of the catheter 2 having the medical agent supplying opening 21 at the tip portion, it is possible to be indwelled accurately in the dermis D.

In addition, also according to the puncture device 1 of such the second exemplified embodiment, it is possible to obtain a similar operation-effect as in a case of the puncture device 1 of the aforesaid first exemplified embodiment.

### THIRD EXEMPLIFIED EMBODIMENT

Next, it will be explained with respect to a third exemplified embodiment of the puncture device 1 of the present invention.

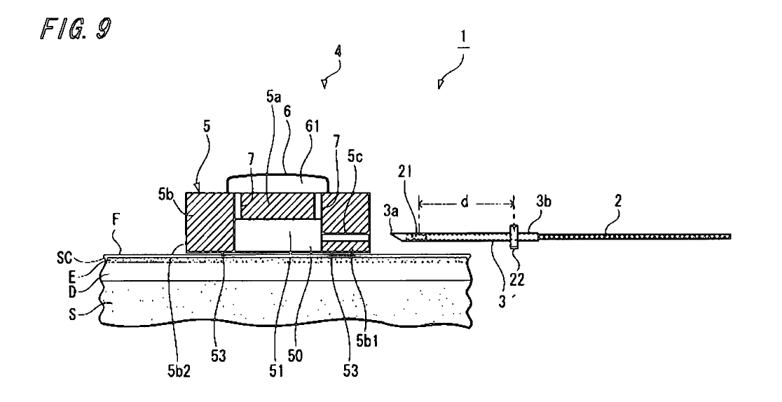

FIG. 9 is a cross-section view showing the third exemplified embodiment of the puncture device 1 of the present invention.

Hereinafter, it will be explained with respect to the puncture device 1 of the third exemplified embodiment mainly about differences from the puncture device of aforesaid first exemplified embodiment and with respect to similar matters, the explanations thereof will be omitted.

In this exemplified embodiment, a puncture needle 3′ is constituted by a needlepoint 3a and a body portion 3b having a hollow portion. Also, there is formed on the body portion 3b along the longitudinal direction with a slit (or slot) for separating the catheter 2 inserted in and attached on the hollow portion.

Also, there is mounted at a position apart from the needlepoint 3a by a predetermined distance d with a ring-shaped positioning member 22 along the circumference surface of the body portion 3b.

There is formed at a tip portion of the catheter 2 with the medical agent supplying opening 21 and the tip portion of the catheter 2 including the medical agent supplying opening 21 is inserted into a hollow portion of the puncture needle 3′.

It should be noted that being different from the first exemplified embodiment, the fixing portion 5 is not provided with a member such as the fixing pin 9 or the like which is engaged with the catheter 2. Also, there is not provided on the peripheral wall portion 5b with the fixing opening 5d.

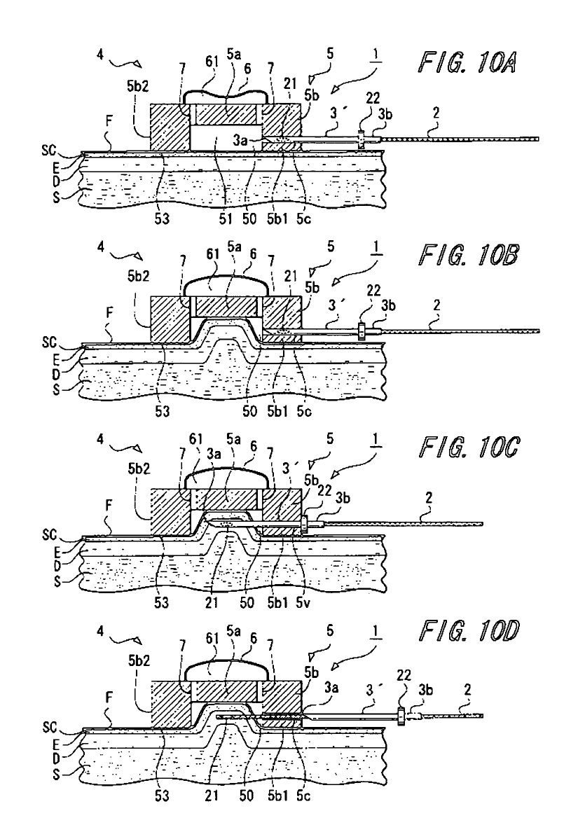

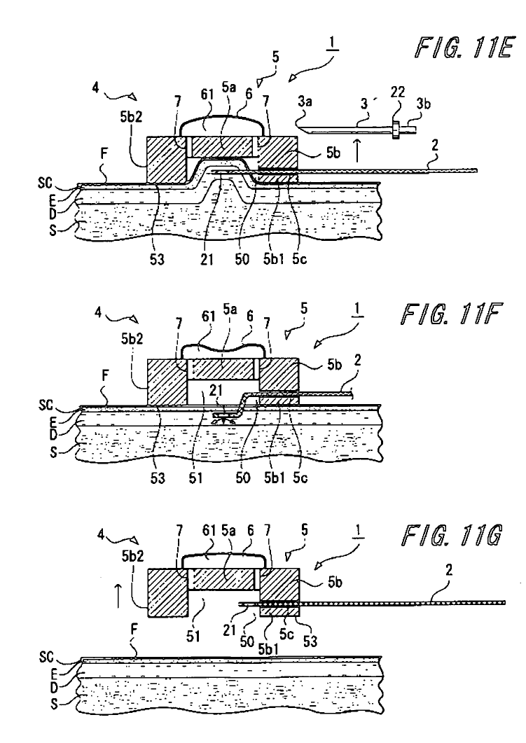

Next, it will be explained by using FIG. 10 and FIG. 11 with respect to usage (operation) of the puncture device 1 of this exemplified embodiment.

First, as shown in FIG. 10(a), the elastic body 6 is deformed by being grasped with fingers or the like and the fixing portion 5 is mounted on the body surface F in a state in which the air in the hollow portion 61 of the elastic body 6 is exhausted.

Concurrently with this, the catheter 2 inserted and attached with the puncture needle 3′ is inserted into the insertion opening 5c formed on the peripheral wall portion 5b of the fixing portion 5. In this case, being different from the first exemplified embodiment, the puncture needle 3′ is retained in the inside of the peripheral wall portion 5b in a state retaining the catheter 2 inside.

Next, as shown in FIG. 10(b), the finger is released from the elastic body 6 and the compressed state of the elastic body 6 is released in which the air in the space 51 of the fixing portion 5 is sucked into the hollow portion 61 through the through-hole 7.

Thereby, the space 51 is depressurized and the body surface F on the inner side of the opening portion 50 is uplifted into the space 51. More specifically, the epidermis E including the stratum corneum SC, the dermis D and the subcutis S are uplifted to the vertical direction with respect to the body surface F. The height of the space 51 is set such that the position of the uplifted dermis D becomes the same height as the position of the insertion opening 5c provided on the peripheral wall portion 5b.

Next, the puncture needle 3′ retaining the catheter 2 inside is inserted into the space 51 and stuck to the uplifted skin.

From this state, as shown in FIG. 10(c), the puncture needle 3′ is inserted into the fixing portion 5 until the positioning ring 22 provided on the puncture needle 3′ is engaged with the outside wall of the peripheral wall portion 5b. At that time, the puncture needle 3′ is stuck to the skin in a state in parallel with the bottom face portion 5a, so that the puncture needle 3′ is stuck in the skin in a state in parallel with the dermis D.

At that time, the medical agent supplying opening 21 provided on the catheter 2 which is retained in the inside of the puncture needle 3′ is positioned in the vicinity of the central portion of the dermis D uplifted in the space 51.

Next, as shown in FIG. 10(d), the puncture needle 3′ projecting from the insertion opening 5c toward the outside of the fixing portion 5 is grasped by fingers or the like and the puncture needle 3′ is pulled out from the insertion opening 5c so as to be moved along the catheter 2. Concurrently with this, in the fixing portion 5, the catheter 2 is separated from the slit formed on the body portion 3b and the catheter 2 is indwelled in the dermis D.

Next, as shown in FIG. 11(e), the puncture needle 3′ is lifted on the outside of the fixing portion 5 to the rear end direction and the puncture needle 3′ is separated from the catheter 2 completely.

Next, as shown in FIG. 11(f), the elastic body 6 is depressed and deformed by a finger or the like concurrently and the air in the hollow portion 61 is sent to the space 51 of the fixing portion 5 through the through-holes 7. Thereby, the space 51 is pressurized and the skin uplifted in the space 51 is deformed so as to be pushed down and the skin is made to return to the original state.

In this state, the medical agent is sent to the medical agent supplying opening 21 from the liquid transmission pump which is connected to the edge portion of the catheter 2 through the hollow portion of the catheter 2. Then, the medical agent is injected into the dermis D from the medical agent supplying opening 21.

After the medical agent is injected into the dermis D, as shown in FIG. 11(g), the catheter 2 is pulled out from the insertion opening 5c and the fixing portion 5 is detached from the body surface F and the operation is finalized.

According to the puncture device 1 of this exemplified embodiment, the mounting and dismounting of the puncture needle 3′ can be carried out through the common insertion opening 5c formed on the fixing portion 5.

In addition, also according to the puncture device 1 of such the third exemplified embodiment, it is possible to obtain a similar operation/effect as in a case of the puncture device 1 of the aforesaid first exemplified embodiment.

It should be noted that the above mentioned exemplified embodiments were explained by using exemplified examples in which the puncture needle is stuck and the indwelling device is indwelled always with respect to the dermis, but it is possible to use them also in case of puncturing an intracutaneous area or a subcutis other than the dermis or further a muscle.

It should be noted that the puncture device of the present invention is not limited by the above-mentioned each exemplified embodiment and besides that, it goes without saying that various modifications or changes can be employed for the materials, the constitutions or the like in the region without departing from the configuration of the present invention.

1. in puncture device puncturing a skin by a puncture needle, the puncture device comprising:
a puncture needle;
an indwelling device indwelled in the skin by said puncture needle;
a skin deforming means for deforming the skin which is provided with a fixing portion having a space in an inside constituted by a bottom face portion and a peripheral wall portion, wherein
an insertion portion for inserting the puncture needle is formed at said peripheral wall portion,
said puncture needle is inserted from said insertion portion, and
the skin deformed into said space by said skin deforming means is to be punctured.

2. The puncture device according to claim 1, wherein said skin deforming means makes deformation so as to uplift said skin.

3. The puncture device according to claim 1, wherein said skin deforming means makes deformation so as to push down said skin.

4. The puncture device according to claim 1, wherein a retaining portion for retaining and fixing the indwelling device is formed at said peripheral wall portion.

5. The puncture device according to claim 4, wherein said retaining portion is formed at a position which becomes symmetrical to said insertion portion.

6. The puncture device according to claim 1, wherein said indwelling device is a catheter.

7. The puncture device according to claim 6, wherein said catheter has at least more than one medical agent supplying opening.

8. The puncture device according to claim 1, wherein said indwelling device is a catheter type detection sensor.

9. In a puncture device puncturing a skin by a puncture needle, the puncture device comprising:
a puncture needle;
an indwelling device indwelled in the skin by said puncture needle;
a fixing portion having a space inside and being constituted by a bottom face portion and a peripheral wall portion; and
a suction means for sucking air of said space, wherein
an insertion opening for inserting the puncture needle is formed at said peripheral wall portion,
said skin is uplifted into said space using said suction means by sucking the air of said space and by depressurizing said space,
said puncture needle is inserted from said insertion opening, and
said uplifted skin is to be punctured.

10. In a puncturing method puncturing a skin by a puncture needle, the puncturing method using:
a puncture needle;
an indwelling device indwelled in the skin by said puncture needle;
a fixing portion having a space in the inside and constituted by a bottom face portion and a peripheral wall portion; and
a skin deforming means for deforming the skin, wherein
said puncture needle is inserted into an insertion portion formed at said peripheral wall portion for inserting a puncture needle, and the skin deformed into said space by said skin deforming means is to be punctured.
